(19) United States
(12) Patent Application Publication
(10) Pub. No.: US 2014/0147527 A1
(43) Pub. Date: May 29, 2014

(54) COMPOSITIONS AND METHODS FOR TREATING EMOTIONAL-PSYCHOLOGICAL STRESS

(71) Applicants: TSUKUBA UNIVERSITY, Tsukuba-shi (JP); INSTITUTE OF ARID REGIONS, TUNISIA, Medenine (TN)

(72) Inventors: Hiroko Isoda, Tsukuba-shi (JP); Junkyu Han, Tsukuba-shi (JP); Mahmoud Ben Othman, Tsukuba-shi (JP); Mohamed Neffati, Medenine (TN)

(73) Assignees: INSTITUTE OF ARID REGIONS, TUNISIA, Medenine (TN); TSUKUBA UNIVERSITY, Tsukuba (JP)

(21) Appl. No.: 13/684,991

(22) Filed: Nov. 26, 2012

(51) Int. Cl. A61K 36/899

(52) U.S. Cl. CPC A61K 36/899
USPC 424/750

(57) ABSTRACT

Cymbopogon schoenanthus is used in folk medicine in North-African for the treatment of rheumatism and to diminish fever. However, whether it possesses anti-stress properties had not previously been established in vivo. Disclosed herein are data demonstrating the effectiveness of C. schoenanthus ethanol extracts on stressors in vitro (heat shock and oxidative stress) and on stressors in vivo (forced swimming test and tail suspension test). Moreover, disclosed are statistically significant anti-stress effects in vivo which occur in a dose-dependent manner. Additionally, the effects of C. schoenanthus ethanol extract exposure is shown to be comparable to a commercially-available antidepressant drug. Accordingly, C. schoenanthus ethanol extracts are useful in new compositions and methods for treating or ameliorating emotional-psychological stress.

### BACKGROUND OF THE INVENTION

Cymbopogon schoenanthus L. Spreng, is an aromatic herb known in Tunisia by the name of “El bekhirai”. Fresh young leaves are consumed in salads and are used in traditional meat recipes. Due to its pleasant aroma and taste, it is used in aromatic teas that is consumed in Northern Africa. Besides its use in culinary, C. schoenanthus is also used in folk medicine. Its medicinal properties were described by “Pliny the Elder” in his book Naturalis Historia, which reports use in the treatment of rheumatism and fever. This author also describes its use as a diuretic, insecticide, and a poultice to cure dromedary wounds. In Southern Tunisia, this plant is used in the treatment of rheumatism and to diminish fever. In North Africa, it is used for treating anorexia. In the Djanet area of Algeria, it is used to restore appetite. The infusions are taken as a diuretic, it cures intestinal troubles and, in the form of decoction, it acts against food poisoning and helps also in digestion.

A number of herbs and dietary supplements have demonstrable effects on mood, memory, insomnia, and reduced appetite. As a result, psychiatric conditions, especially depression, are commonly treated with complementary and alternative therapies.

Nonetheless, it is unknown whether ethanol extracts of C. schoenanthus have in vitro and in vivo anti-stress activities. Furthermore, it has not been demonstrated that C. schoenanthus has anti-emotional-psychological stress activities. If its has such activities, then extracts of C. schoenanthus would be useful in new methods for treating or ameliorating emotional-psychological stress.

### SUMMARY OF THE INVENTION

As described below, the present invention features compositions including a Cymbopogon schoenanthus ethanol extract and methods for treating or ameliorating emotional-psychological stress by administering a composition including a C. schoenanthus ethanol extract.

In one aspect, the invention provides a method for treating or ameliorating emotional-psychological stress in a subject in need thereof involving administering to the subject a composition comprising a C. schoenanthus ethanol extract as an active ingredient. An effective amount of the composition is administered as necessary to treat or ameliorate symptoms relative to an untreated patient. Emotional-psychological stress includes one or more of the following symptoms: abnormal blood pressure, agitation, anger, anxiety, confusion, depression, distractibility, exhaustion, fear, insecurity, insomnia, irritability, lack of sexual desire, loss of appetite, low self esteem, menstrual issues, migraine, mood swings, nervousness, panic attacks, rashes, sexual dysfunction, skin eruptions, and social withdrawal. In certain embodiments, the ethanol extract is obtained from a root, a stem, or a blade from a C. schoenanthus plant. In some embodiments, the composition is formulated as a food product which is a beverage, a bread, a candy, a cereal, a chocolate, a coffee, a condiment, a cookie, a cracker, an energy drink, a gel, an ice cream, a jelly, a juice, a milk-containing beverage, a nutritional bar, a pasta, a paste, a processed fruit, a processed grain, a processed meat, a processed vegetable, a pudding, a snack bar, a soft drink, a tea, or a yogurt. In certain embodiments, the composition is formulated as a dietary supplement which is in the form of a capsule, a concentrate, a dry syrup, a granule, a liquid gel, a liquid, a lozenge, a paste, a pill, a powder, a softgel, a syrup, or a tablet. In some embodiments, the C. schoenanthus ethanol extract can be between 1/10 and 1/10,000 of the composition, e.g., between 1/100 and 1/2,000, between 1/500 and 1/1,000, 1/500, 1/750, and 1/1,000 of the composition. In certain embodiments, the C. schoenanthus ethanol extract is evaporated to produce a dried ethanol extract C. schoenanthus before being included in the composition. In some embodiments, the method uses a composition including between 1 mg and 1,000 mg of the dried C. schoenanthus ethanol extract per kg of the subject, e.g., between 5 mg and 500 mg; 10 mg and 300 mg; 25 mg and 200 mg; and 50 mg and 100 mg of the dried C. schoenanthus ethanol extract per kg of the subject. The subject can be a human.

In another aspect, the invention provides a method for treating or ameliorating emotional-psychological stress in a human in need thereof involving administering to the human a tea comprising a dried C. schoenanthus ethanol extract as an active ingredient.

The above aspects and embodiments can be combined in an possible manner.

Other features and advantages of the invention will be apparent from the detailed description and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “C. schoenanthus ethanol extract” is meant one or more compounds which are normally present in a sample of C. schoenanthus and which can be extracted, isolated, or solubilized from the sample of C. schoenanthus. The C. schoenanthus ethanol extract can be a first product obtained by combining a dried C. schoenanthus sample with ethanol; thus, the one or more compounds can remain in an ethanol solution. Alternately, the first product obtained by combining a dried C. schoenanthus sample with ethanol can be evaporated to produce a dried product; thus, the C. schoenanthus ethanol extract may contain the one or more compounds but substantially no ethanol. Alternately, ethanol and/or the one or more compounds can be removed from the first product using other methods known in the art.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. “

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of a compound, molecule, nucleic acid, or protein to be detected.

By “disease” is meant any condition or disorder that damages or interferes with the normal function of a cell, tissue, or organ.

By “effective amount” is meant the amount of a required to ameliorate the symptoms of a disease relative to an untreated patient. The effective amount of active ingredient(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “extracted”, “isolated,” “purified,” or “biologically pure” refer to material that is free to varying degrees from components which normally accompany it as found in its native state. “Extract” and “isolate” denote a degree of separation from original source or surroundings. “Purify” denotes a degree of separation that is higher than isolation. A “purified” or “biologically pure” compound is sufficiently free of other materials such that any impurities do not materially affect the biological properties of the compound or cause other adverse consequences. That is, a compound is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography.

By an “isolated compound” is meant a compound that has been separated from components that naturally accompany it. Typically, the compound is isolated when it is at least 60%, by weight, free from the naturally-occurring organic molecules with which it is naturally associated. Preferably, the preparation is at least 75%, more preferably at least 90%, and most preferably at least 99%, by weight, of a desired compound. An isolated compound of the invention may be obtained, for example, by extraction from a natural source, by expression of a recombinant nucleic acid encoding such a compound, or by chemically synthesizing the compound.

As used herein, “obtaining” includes collecting, harvesting, purchasing, synthesizing, or otherwise acquiring matter.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition.

By “subject” is meant a mammal, including, but not limited to, a human or non-human mammal, such as a bovine, equine, canine, ovine, feline, or rodent.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Ranges from 50 to 100 include similar steps. Ranges between 100 and 200, 200 and 300, 300 and 400, and so forth also include similar steps.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any composition or method provided herein can be combined with one or more of any of the other compositions and methods provided herein.

### BRIEF DESCRIPTION OF THE DRAWINGS

FIG. 1 is a graph showing the effects of Cymbopogon schoenanthus ethanol extract exposure on SH-SY5Y cell viability.

FIGS. 2A and 2B are graphs showing the effects of C. schoenanthus ethanol extract exposure on (A.) HSP47 expression and (B.) cell viability in Chinese Hamster Ovary cells.

FIGS. 3A and 3B are graphs showing the effects of C. schoenanthus ethanol extract exposure on (A.) choline (“CH”) and (B.) acetylcholine (“ACH”) amount in SH-SY5Y cells after stress (heat shock).

FIGS. 4A-4D are graphs showing the effects of C. schoenanthus ethanol extract exposure on H₂O₂-induced cell death in SH-SY5Y cells.

FIG. 5 is a graph showing the effects of C. schoenanthus ethanol extract exposure on H₂O₂-induced stress response (increase in heat shock protein expression) in SH-SY5Y cells.

FIGS. 6A and 6B are graphs showing the effects of C. schoenanthus ethanol extract exposure on body weight of mice subjected to (A.) a forced swim test (FST) or subjected to (B.) a tail suspension test (TST).

FIG. 7 is a graph showing the effects of C. schoenanthus ethanol extract exposure on immobility time in mice in a tail suspension test (TST).

FIG. 8 is a graph showing the effects of C. schoenanthus ethanol extract exposure on immobility time in mice in a forced swim test (FST).

### DETAILED DESCRIPTION OF THE INVENTION

The invention features compositions and methods that are useful for treating or ameliorating emotional-psychological stress.

The invention is based, at least in part, on the following discoveries. First, Cymbopogon schoenanthus extracts have anti-stress effects in vitro. In particular, C. schoenanthus extracts inhibited heat shock protein activation and had a neuroprotective effect against hydrogen peroxide-induced cytotoxicity in human neuronal SH-SY5Y cells. Second, C. schoenanthus extracts have anti-stress effects in vivo. In particular, C. schoenanthus extracts administered to mice had significantly improved performance in forced swim test (FST) and tail suspension test (TST) assays, two assays that are predictive of anti-depressant activity. These discoveries indicate that C. schoenanthus possesses antioxidant and anti-stress properties and can be used for treating or ameliorating symptoms of emotional-psychological stress.

Emotional-psychological stress, commonly known as “stress”, typically describes a negative concept that can have an impact on one's mental and physical well-being. Signs of stress may be cognitive, emotional, physical, and/or behavioral. Emotional-psychological stress disorders include but are not limited to: Acute stress reaction, Depression, General adaptive syndrome, Generalized anxiety syndrome, Panic disorder, Phobia, and Post-traumatic stress disorder (PTSD). Disorders may be related to neurological or neurodegenerative disorders, e.g., Alzheimer's disease, Huntington's disease, Parkinson's disease, stroke, and trauma. Disorders may be hormonal in nature, e.g., Addison's disease, post-partum depression, and premenstrual syndrome. Symptoms of emotional-psychological stress include but are not limited to abnormal blood pressure, agitation, anger, anxiety, confusion, depression, distractibility, exhaustion, fear, insecurity, insomnia, irritability, lack of sexual desire, loss of appetite, low self esteem, menstrual issues, migraine, mood swings, nervousness, panic attacks, rashes sexual dysfunction, skin eruptions, or social withdrawal. Other causes, conditions, or symptoms of emotional-psychological stress are described in the Diagnostic and Statistical Manual of Mental Disorders published by the American Psychiatric Association (current edition is DSM-IV-TR); the entire contents of which is herein incorporated by reference.

The therapeutic methods of the disclosure, which include prophylactic treatments, which, in general, comprise administration of a therapeutically effective amount of a composition described herein, to a subject (e.g., animal and human) in need thereof, including a mammal, particularly a human to produce a desired effect, i.e., treating or ameliorating emotional-psychological stress. Such treatment will be suitably administered to subjects, particularly humans, suffering from, having, susceptible to, or at risk for an emotional-psychological stress or having symptoms thereof. Identifying a subject in need of such treatment or determination of those subjects “at risk” can be in the judgment of a subject or a health care professional or veterinarian and can be subjective (e.g., opinion) or objective (e.g., measurable by a test, diagnostic method, family history, and the like).

Administration of a composition can be accomplished via a single or divided doses.

As defined herein, a therapeutically effective amount of composition (i.e., an effective dosage) depends on the formulation selected. The formulations can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present.

Human dosage amounts can initially be determined by extrapolating from the amount of compound used in animal models (e.g., mice), as a skilled artisan recognizes it is routine in the art to modify the dosage for humans compared to animal models.

In certain embodiments it is envisioned that the dosage may vary from between about 1 mg of an evaporated C. schoenanthus ethanol extract/kg body weight to about 1000 mg of an evaporated C. schoenanthus ethanol extract/kg body weight; or from about 5 mg/kg body weight to about 500 mg/kg body weight or from about 10 mg/kg body weight to about 300 mg/kg body weight; or from about 25 mg/kg body weight to about 200 mg/kg body weight; or from about 50 mg/kg body weight to about 100 mg/kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000 mg/kg body weight.

The weight (in mg) of a C. schoenanthus extract represents the weight of dried C. schoenanthus sample originally used to prepare a first product obtained by combining a dried C. schoenanthus sample with ethanol. Alternately, the weight (in mg) of a C. schoenanthus extract represents the weight of a dried product that is produced by evaporating (or otherwise excluding ethanol from) the first product by combining a dried C. schoenanthus sample with ethanol. Additionally, the weight (in mg) of a C. schoenanthus extract represents the weight of a dried fractionated product which is produced when a C. schoenanthus extract is further purified into a plurality of distinct fractions thereof.

In other embodiments, a C. schoenanthus ethanol extract is not evaporated and the active ingredients remain in solution with ethanol. In these embodiments, to produce a composition, the ethanol extract is diluted in water or other suitable liquids to achieve a desired final concentration. For example, when 1 part C. schoenanthus ethanol extract is diluted in 99 parts liquid, it can be said that the C. schoenanthus ethanol extract comprises 1/100 of the composition. The C. schoenanthus ethanol extract can comprise 1/1, 1/2, 1/3, 1/4, 1/5, 1/6, 1/7, 1/8, 1/9, 1/10, 1/15, 1/20, 1/25, 1/30, 1/40, 1/60, 1/70, 1/80, 1/90, 1/100, 1/200, 1/250, 1/300, 1/400, 1/500, 1/600, 1/700, 1/750, 1/800, 1/900, 1/1000, 1/1500, 1/2000, 1/3000, 1/4000, 1/5000, 1/6000, 1/7000, 1/8000, 1/9000, or 1/1000 of a composition.

Of course, the above-mentioned dosage amounts may be adjusted upward or downward, as is routinely done in such treatment protocols, depending on the results of the initial clinical trials and the needs of a particular patient.

Toxicity and therapeutic efficacy of such formulations can be determined by standard procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Formulations which exhibit high therapeutic indices can be preferred. While formulations that exhibit toxic side effects may be used, care should be taken to design a delivery system that targets such formulations to the site of affected tissue in order to minimize potential damage to uninfected cells and, thereby, reduce side effects.

The data obtained from the cell culture assays and animal studies can be used in formulating a range of dosage for use in humans. The dosage of such formulations optionally lies within a range of circulating concentrations that include the ED₅₀ with little or no toxicity. The dosage may vary within this range depending upon the dosage form employed and the route of administration utilized. For any formulation used in a method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulations suitable for oral administration can consist of, e.g., (a) liquid solutions, such as an effective amount of the packaged cargo (e.g., nucleic acid) suspended in diluents such as water, saline, or PEG 400; (b) capsules, sachets, or tablets, each containing a predetermined amount of the cargo, as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and compatible carriers. Lozenge forms can comprise the cargo in a flavor, e.g., sucrose, as well as pastilles comprising the cargo in an inert base, such as gelatin and glycerin or sucrose and acacia emulsions, gels, and the like containing, in addition to the cargo, carriers known in the art.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The practice of the present invention employs, unless otherwise indicated, conventional techniques of biochemistry, botany, cell biology, food science, immunology, microbiology, molecular biology (including recombinant techniques), and nutrition which are well within the purview of the skilled artisan. Such techniques are explained fully in the literature.

### EXAMPLES

The present invention is described by reference to the following Examples. The following examples provide those of ordinary skill in the art with a complete disclosure and description of how to make and use the therapeutic methods of the invention, and are not intended to limit the scope of what the inventors regard as their invention. Standard techniques well known in the art or the techniques specifically described below were utilized.

### Example 1

### Exposure to C. Schoenanthus Ethanol Extracts does not Reduce Viability of SH-SY5Y Cells

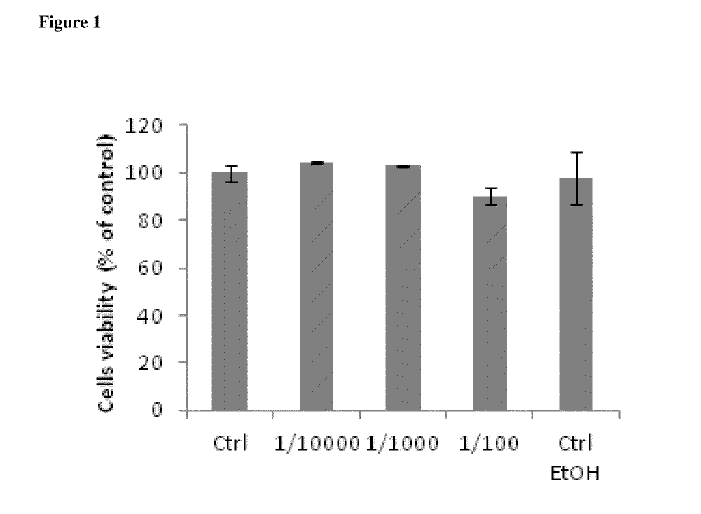

To determine the concentration of C. schoenanthus ethanol extracts which can be used to investigate their anti-stress effect, SH-SY5Y cells were cultured with several concentrations (1/100, 1/1000, or 1/10000 v/v dilution of C. schoenanthus ethanol extract in culturing medium) for 72 hours. As shown in FIG. 1, none of the C. schoenanthus ethanol extracts concentrations were toxic to the SH-SY5Y cells. Actually, cell viability increased slightly compared to controls for 1/1,000 and 1/10,000 concentrations, although cell viability was slightly reduced when cells were treated with the 1/100 concentration.

### Example 2

### In a Dose-Dependent Manner, Exposure to C. Schoenanthus Ethanol Extracts Reduces HSP47 Expression in Heat-Shocked Chinese Hamster Ovary Cells

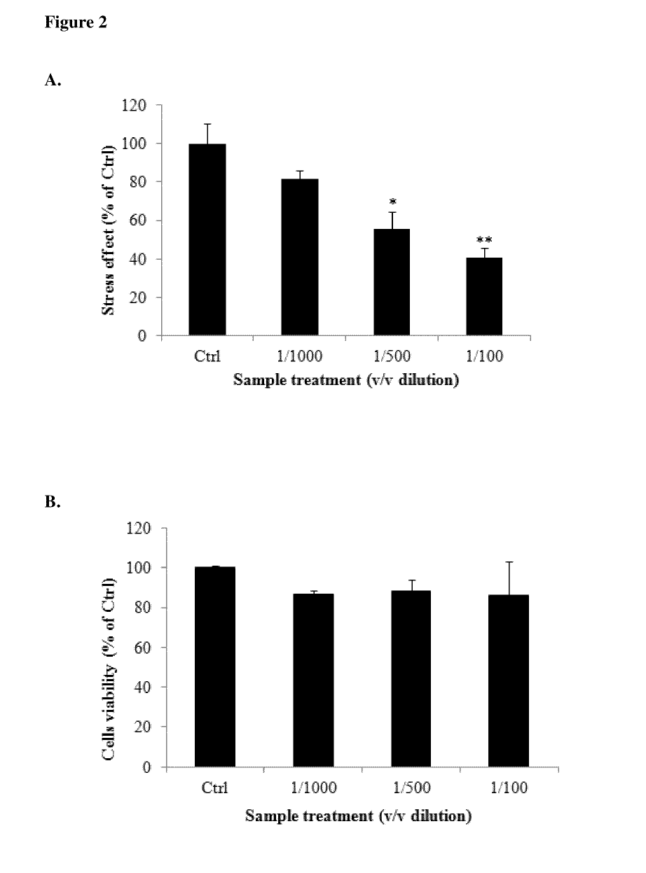

To investigate the anti-stress effect of C. schoenanthus extracts, Chinese Hamster Ovary (CHO) cells were stably transfected with a construct comprising the Heat Shock Protein 47 (HSP47) promoter linked to the coding sequence for β-galactosidase. These cells were plated at 1×10⁵ cells/ml and were stressed by heat shock for 90 min at 42° C., 5% CO₂. After a two-hour recovery, cells were treated with C. schoenanthus ethanol extracts of different concentrations (1/100, 1/500, or 1/1000 v/v in medium) for three hours. FIG. 2A shows that HSP47 expression is significantly reduced in a dose-dependent manner. As shown in FIG. 2B, the viability of CHO cells was not reduced by treatment with C. schoenanthus ethanol extracts.

### Example 3

### Exposure to C. Schoenanthus Ethanol Extracts Increases Acetylcholine Levels in Heat-Shocked SH-SY5Y Cells

There are direct relationships between stress and neurotransmitter (e.g., Acetylcholine, Dopamine, Norepinephrine, Glutamate, and Serotonin) release. Additionally, there is a link between acetylcholine and Alzheimer's disease: there is approximately a 90% loss of acetylcholine in the brain of one suffering from Alzheimer's disease.

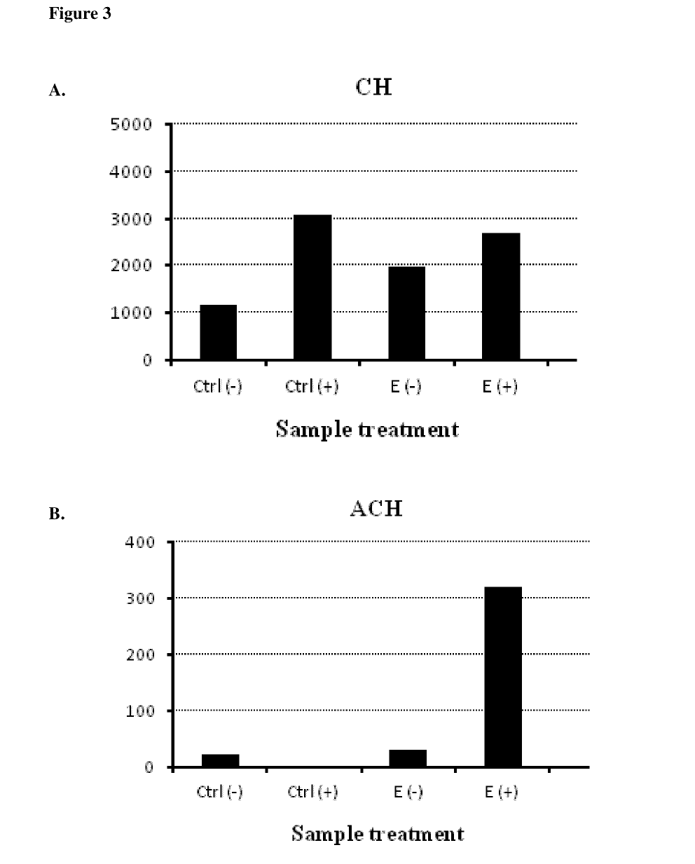

As shown in FIG. 3A, levels of choline (“CH”) appear to be unaffected by heat shock and/or exposure to C. schoenanthus ethanol extracts. In contrast, as seen in FIG. 3B (“E(+)”), exposure to C. schoenanthus ethanol extracts (at a 1/1000 v/v dilution in medium) increases acetylcholine (“ACH”) levels in heat-shocked SH-SY5Y cells. Cells that were not heat shocked or exposed to ethanol extracts (“Ctrl(−)”); cells that were heat shocked but not exposed to ethanol extracts (“Ctrl(+)”), and cells that were not heat shocked cells but were exposed to ethanol extracts (“E(−)”) did not have increases in acetylcholine levels.

### Example 4

### Exposure to C. Schoenanthus Ethanol Extracts Reduces H₂O₂-Induced Cell Death in SH-SY5Y Cells

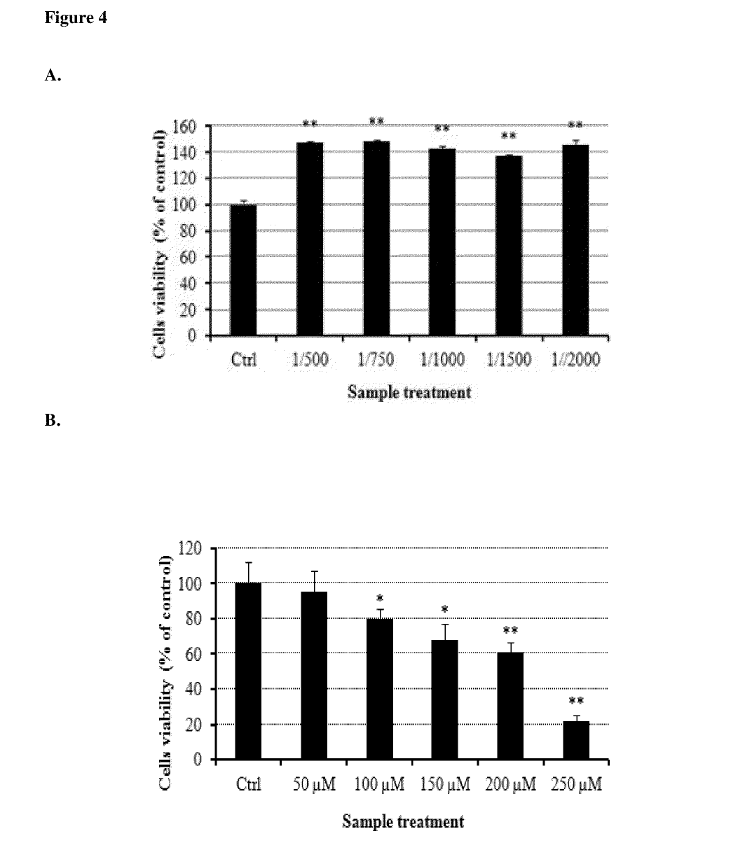

SH-SY5Y cells were treated with different concentrations of C. schoenanthus extract (at a 1/500, 1/750, 1/1000, 1/1500, or 1/2000 v/v dilution in medium) for 72 hours and cell viability was determined by MTT assay. As shown in FIG. 4A, viability of SH-SY5Y cells was increased when exposed to C. schoenanthus ethanol extracts when compared to the untreated control cells.

To evaluate whether H₂O₂ influences neuronal cytotoxicity, SH-SY5Y cells were treated with various concentrations of H₂O₂ (0, 50, 100, 150, 200, or 250 μM) for 24 hours. As shown in FIG. 4B, H₂O₂ induced a dose-dependent cytotoxicity in SH-SY5Y cells.

To determine whether C. schoenanthus ethanol extracts have protective effects against H₂O₂-induced reduction in cell viability, SH-SY5Y cells were exposed to 1/500, 1/1000, or 1/2000 v/v dilutions in medium of C. schoenanthus ethanol extract for 12 hours or for 24 hours, followed by treatment with 150 μM of H₂O₂ for 24 hours. As shown in FIGS. 4C and 4D, the reduction of cell viability caused by H₂O₂ treatment was significantly attenuated by exposure to C. schoenanthus ethanol extracts. The positive effect of C. schoenanthus ethanol extracts was directly correlated with how long cells were exposed to the ethanol extracts, since the cells exposed for 24 hours (FIG. 4D) had increased cell viability when compared to cells exposed for 12 hours (FIG. 4C).

### Example 5

### Exposure to C. Schoenanthus Ethanol Extracts Reduces H₂O₂-Induced Heat Shock Protein Expression in SH-SY5Y Cells

To investigate the effects of C. schoenanthus ethanol extract on heat shock protein expression, SH-SY5Y cells were exposed to different concentrations of C. schoenanthus ethanol extract (at 1/500, 1/1000, or 1/2000 v/v dilution in medium) for 72 hours and were then treated with 150 μM H₂O₂ for 24 hours. Control cells did not receive H₂O₂ treatments; “H₂O₂” cells received H₂O₂ treatments but were not exposed to C. schoenanthus ethanol extracts.

Total RNA was extracted from the SH-SY5Y using the ISOGEN PB Kit and Real Time PCR was performed.

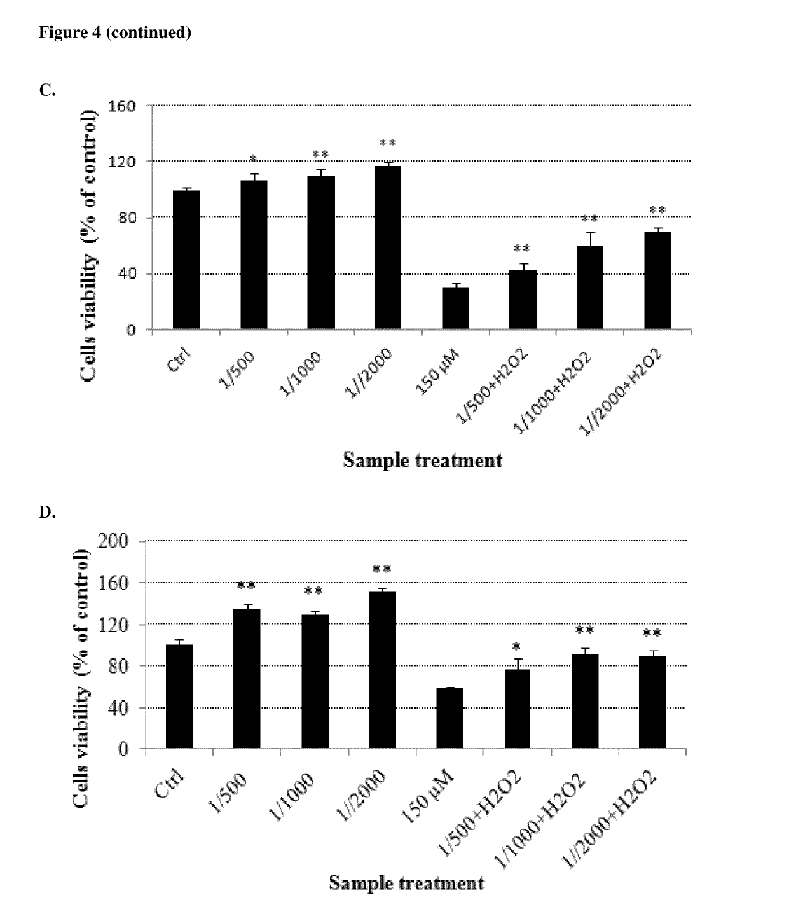

As shown in FIG. 5, exposure to C. schoenanthus ethanol extracts reduced H₂O₂-induced expression of heat shock protein 27 (“HSP27”) and heat shock protein 90 (“HSP90”) in SH-SY5Y cells.

### Example 6

### Oral Administration of C. Schoenanthus Ethanol Extracts does not Reduce Body Weight in Mice Assayed by Forced Swim Test (FST) or Tail Suspension Test (TST)

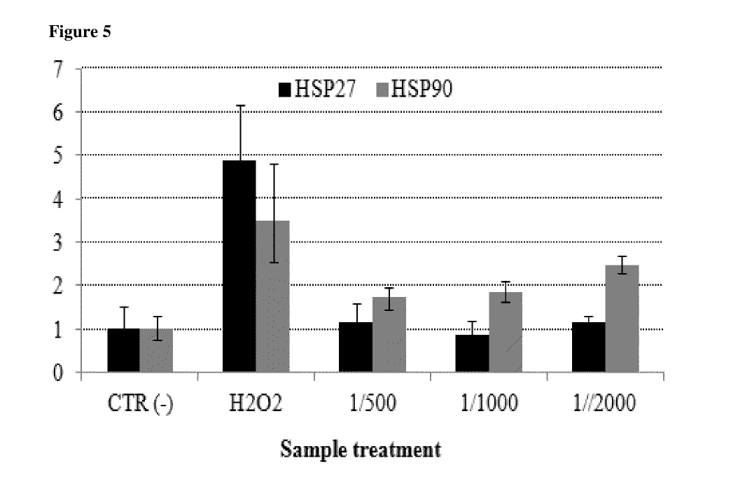

Oral administration of C. schoenanthus ethanol extracts at 100 or 200 mg/kg of animal body weight did not cause the death of any animal or any change in an animal's hair color. In addition, as shown in FIGS. 6A and 6B, oral administration of C. schoenanthus ethanol extracts at these doses did not result in a significantly reduced body weight in mice assayed by TST or FST, when compared to control mice.

Accordingly, in vivo exposure to C. schoenanthus ethanol extracts does not negatively affect the health of an exposed animal.

### Example 7

### Oral Administration of C. Schoenanthus Ethanol Extracts Reduces Behavioral Despair in Mice Assayed by Tail Suspension Test (TST)

In TST, mice are forced to hang in a confined space from which they cannot escape; this causes the mice to exhibit a characteristic behavior of immobility. This immobility, referred to as behavioral despair, reproduces a condition similar to human depression. Thus, a reduction in the total duration of immobility indicates an anti-emotional-psychological stress effect and accordingly, an anti-depressive effect.

The effects of oral administration of C. schoenanthus ethanol extracts and imipramine (“Imp”; a well-known anti-depression drug) on the immobility time in the TST were investigated.

Mice were orally administered C. schoenanthus ethanol extracts at 100 or 200 mg/kg of animal body weight, imipramine at 15 mg/kg of animal body weight, or distilled water for thirteen days.

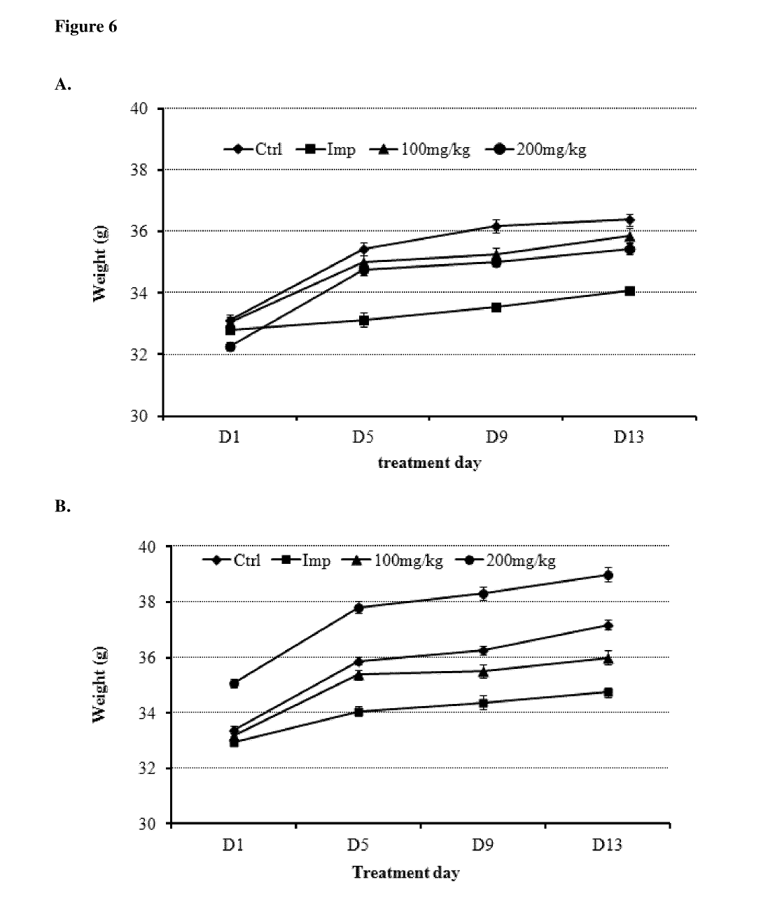

As shown in FIG. 7, starting after one day of oral administration, C. schoenanthus ethanol extracts reduced immobility time in TST more effectively than imipramine.

Accordingly, in vivo exposure to C. schoenanthus ethanol has an anti-emotional-psychological stress/anti-depression effect on an exposed animal.

### Example 8

### Oral Administration of C. Schoenanthus Ethanol Extracts Reduces Behavioral Despair in Mice Assayed by Forced Swim Test (FST)

In FST, mice are forced to swim in a confined space from which they cannot escape; this causes the mice to exhibit a characteristic behavior of immobility. This immobility, referred to as behavioral despair, reproduces a condition similar to human depression. Thus, a reduction in the total duration of immobility indicates an anti-emotional-psychological stress effect and accordingly, an anti-depressive effect.

Mice were orally administered C. schoenanthus ethanol extracts at 100 or 200 mg/kg of animal body weight, imipramine at 15 mg/kg of animal body weight, or distilled water for thirteen days.

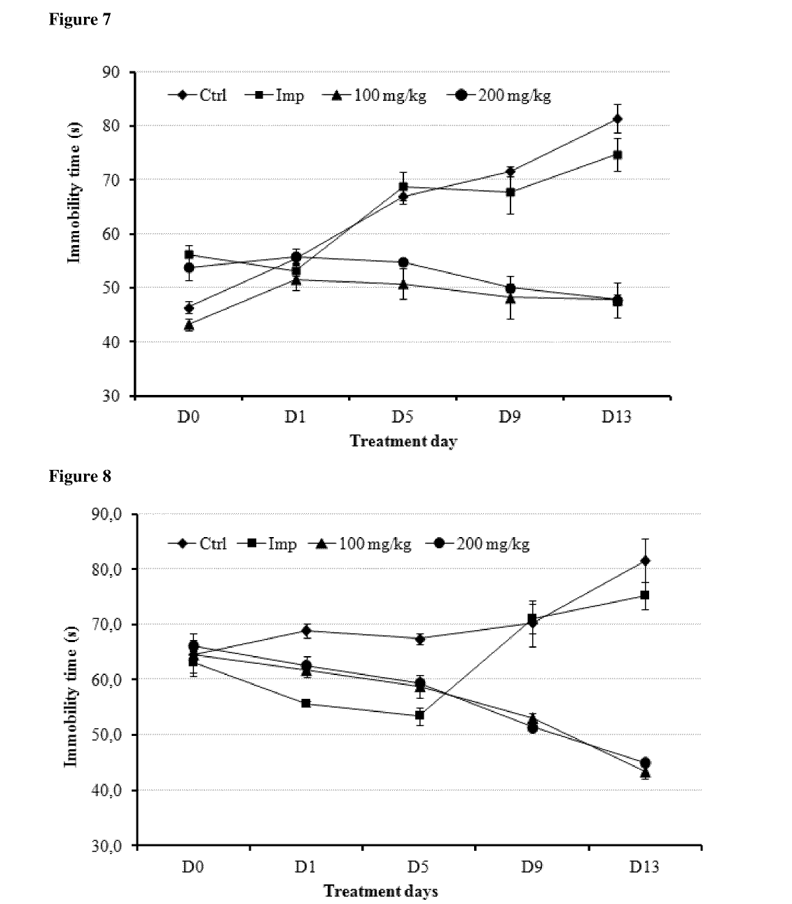

As shown in FIG. 8, after five days of oral administration, C. schoenanthus ethanol extracts reduced immobility time in FST more effectively than imipramine. Indeed, C. schoenanthus ethanol extracts significantly and progressively decreased immobility time over the duration of the experiment. The effect of extract appeared highest after 13 days of treatment, resulting in 46.8% (100 mg/kg) and 46% (200 mg/kg) immobility reduction.

Accordingly, in vivo exposure to C. schoenanthus ethanol has an anti-emotional-psychological stress/anti-depression effect on an exposed animal.

Stress is a state of psychological and/or physiological imbalance resulting from many factors in human's life, such as challenges, overwork, fatigue, and so forth.

The foregoing demonstrates the effectiveness of exposure to Cymbopogon schoenanthus ethanol extracts on stressors in vitro (heat shock and oxidative stress) and on stressors in vivo (forced swimming test and tail suspension test).

Heat shock proteins (HSPs) play important roles in cells. They have become therapeutic targets in research of neurodegenerative disease and of aging because the pathogenic mechanisms of these diseases is thought to be related to an abnormal increase in improperly folded proteins. It has been reported that HSP27 protects cells from protein aggregation and sequesters cytochrome C when released from the mitochondria into the cytosol; it also has been shown to reduce the production of reaction oxygen species (ROS). HSPs are strongly induced by different stresses such as heat, irradiation, oxidative stress, or anticancer chemotherapy. Experimental depletion of HSP27 suggests that HSP27 mainly functions as an inhibitor of caspase activation. Knock-down of HSP27 by small interfering RNAs induces apoptosis through caspase-3 activation. HSP27 also has antioxidant properties which may be related to its ability to maintain glutathione in its reduced form and to decrease the abundance of reactive oxygen species. These anti-oxidant properties of HSP27 seem particularly relevant for HSP27-mediated cytoprotection in neuronal cells and involve phosphorylated HSP27.

The data presented in the above Examples (which show that cells treated H₂O₂ have an increase in HSP27 and HSP90 expression when compared to cells previously exposed to C. schoenanthus ethanol extracts) is consistent with the above-described role of HSP27.

The above-presented data also demonstrates that a stress (heat shock) decreases acetylcholine levels in SH-SY5Y cells. However, when cells were exposed to C. schoenanthus extract after heat shock, there was a significant increase of acetylcholine levels. This increase in acetylcholine has as role in defense against the stress related to heat shock. This is consistent with other's findings that in response to a stressful event, acetylcholine release is significantly increased in the prefrontal cortex and hippocampus.

Studies of stress and treatments for stress in humans are challenging using model organisms. This is because it is difficult for a researcher/clinician to characterize or quantify a type of stress and its severity since stress is often subjective and requires the stressed individual to verbally describe his/her stress. Clearly this is not possible with model organisms. Thus, the methods described above use generally-accepted in vitro and in vivo experiments that provide insight into human stress. For example, the tail suspension test (TST) and forced swimming test (FST) are the most commonly-used predictive tests for screening the anti-stress effects of medicinal plants. In these tests, animals are placed under stress from which they cannot escape and the animal's biological response (immobility) can be quantified by a researcher.

C. schoenanthus, is an herbal medicine widely used in North-African for the treatment of rheumatism and to diminish fever. However, its anti-stress property had not yet been established in vivo.

The above data is the first demonstration that oral administration of a C. schoenanthus extract (at 100 and 200 mg/kg) had an anti-stress effect in either mice subjected to TST or FST, as demonstrated by a significant reduction of immobility time. Moreover, the reduction of immobility time due to exposure to C. schoenanthus was greater than that for imipramine (at 15 mg/kg), a classical tricyclic antidepressant.

In the above-described in vivo experiments, mice are forced into situations from which they cannot escape and exhibit a characteristic behavior of immobility. This immobility, referred to as behavioral despair in animals, reproduces a condition similar to human depression (Renard et al., 2003). Thus, a reduction in the total duration of immobility indicates an anti-stress effect (Cryan et al., 2005; Steru et al., 1985; Willner, 1984).

In recent decades, there has been increasing interest in Western Medicine for phytochemical combinations, which have been fundamental in traditional systems of herbal medicine. Combinations of some compounds present in a plant synergistically increase the herbal medicine's therapeutic activity. Although not bound by theory, the C. schoenanthus extracts disclosed herein likely have superior activity due to synergy brought about by the combination of compounds included in an extract.

The present results disclose that C. schoenanthus extracts are effective in producing significant anti-stress effects in these animal models. Moreover, the anti-stress effects observed was dose dependent in two in vivo assays.

Accordingly, C. schoenanthus extract is shown herein to have an in vivo, dose-dependent anti-emotional-psychological stress/anti-depression effects that are comparable to a commercially-available antidepressant drug.

The results reported above were obtained using the following methods and materials.

### Preparation of C. Schoenanthus Ethanol Extracts

Dried C. schoenanthus leaves were ground into fine powder. One hundred grams of the powdered leaves was soaked in 1000 ml of 70% ethanol for two weeks. Subsets of the ethanol extracts were evaporated by standard methods and other subsets were not evaporated. The evaporated subsets were dissolved or suspended in distilled water and the not-evaporated subsets were diluted in distilled water.

### Cell Culture

Two types of cells were used: human neuroblastoma SH-SY5Y cells and Chinese hamster ovary (CHO) cells. SH-SY5Y were cultured in 100 cm² dishes with media including Dulbecco's minimum essential medium (DMEM; Sigma, USA) and Ham's F-12 nutrient mixture (Sigma, USA) supplemented with 15% fetal bovine serum (FBS; Sigma, USA), 1% non-essential MEM amino acid and 1% penicillin (5000 μg/ml)-streptomycin (5000 IU/ml) solution (ICN Biomedicals, Inc.) at 37° C. in a 95% humidified air-5% CO₂ incubator. CHO cells were stably transfected with (+) or without (−) a construct comprising the Heat Shock Protein 47 (HSP47) promoter connected to the structural gene for β-galactosidase. The cells were cultured in 75 cm² flasks with: F12 medium, 10% fetal bovine serum, 0.2% kanamycin solution and 0.1% of G418 (Gibco® BRL 13075-015) at 37° C. in 95% humidified air-5% CO₂ incubator.

### Cell Viability Assay

A cell survival analysis was performed using 3-(4,5-Dimethylthiazol-2-yl)-2,5-diphenyltetrazolium bromide (MTT), which was dissolved in PBS to a final concentration of 5 mg/ml. After a 72-hour incubation of cells with different concentrations of C. schoenanthus extract, 10 μl of the MTT solution was added to each well of 96-well plates and incubated for six hours at 37° C. in a 95% humidified air-5% CO₂ incubator. Then, 100 μl of 10% of sodium dodecyl sulfate (SDS) was added and after overnight incubation, the absorbance was determined at 570 nm. The control wells which contained cultured cells with medium only were considered as 100% cell viability.

### HSP47 Assay

Two 96-well plates were used in this assay: one for HSP47 (−) cells and another for HSP47 (+) cells (as defined above), each one contained 1×10⁴ cells/well in 100 μl of culture medium. After a 48-hour incubation in a 95% humidified air-5% CO₂ incubator at 37° C., the cells were subjected to heat shock for 90 min at 42° C., then incubated at 37° C. for two hours (a recovery time). After the recovery time, cells were treated with extracts for three hours in a 95% humidified air-5% CO₂ incubator at 37° C. After incubation, cells were washed twice with PBS, and 50 μl of lysis buffer was added, then the plates were incubated for 30 min at room temperature (RT). Carefully, cells were mixed up and 20 μl of cell lysate was transferred to a black plate, to which 100 μl of substrate solution (10 mM NaH₂—PO₄ 2H₂O, 100 mM NaCl, 1% 4-methylumbelliferyl-β-D-galactopyranoside (MUG), pH 7) was added in order to trigger the conversion of MUG into galactose and methylumbelliferyl by galactosidase. After 30 min at RT, 60 μl of reaction stop buffer (1 M glycine-NaOH, pH 10.3) was added and the fluorescence at 365 nm excitation/450 nm emission was then determined using a multi-detection microplate.

### Acetylcholine Analysis

To show the effects of a sample extract on acetylcholine amount in heated cells (heat shock stress), 3×10⁵ cells/ml were plated per 100 mm dish. The cells were treated to 24 hours at 37° C. cells and then were heat shocked at 42° C. for 90 min, then, after the medium was removed and washed with Opti-MEM®, 10 ml of Opti-MEM° with sample extracts were added.

Extraction of Acetylcholine (ACh) was done after a six-hour incubation at 37° C. The cells were washed with PBS and then 1 ml of 15% Formic acid in Acetone was added. The cells were scraped from the dishes and the cells were collected and sonicated. After sonication, the cell sonicates were centrifuge at 12,000 rpm for 15 min. The supernatant was collected and dried by speed vacuum for six hours. 1 ml of Milli-Q water was added to the dried product and the solution was filtered (0.22 μm) before HPLC analysis.

### Cell Viability Assay for Neuroprotective Effect of C. Schoenanthus Against H₂O₂-Induced Cytotoxicity in SH-SY5Y Cells

SH-SY5Y cells were treated with H₂O₂ at concentrations ranging from 50 to 250 μM for 24 hours. In preliminary experiments, a concentration of 150 μM H₂O₂ was selected to evaluate the neuroprotective effects of C. schoenanthus on cell viability. C. schoenanthus was added to the medium 12 or 24 hours before treatment with H₂O₂. Each treatment protocol was performed in triplicate. H₂O₂ was freshly prepared from stock solution prior to each experiment.

### Assay for Protective Effects of C. Schoenanthus on H₂O₂-Induced Cell Death

Cells were pretreated with various concentrations (1/500, 1/1000, or 1/2000) of ethanol extract of C. schoenanthus for 72 hours, followed by exposure to 150 μM of H₂O₂ (to produce oxidative stress) in the presence of the same concentrations of C. schoenanthus ethanol extract for 24 hours. H₂O₂ was freshly prepared from stock solution prior to each experiment. Control cells received medium lacking H₂O₂ and a C. schoenanthus extract.

### Assay for the Effects of C. Schoenanthus on H₂O₂-Induced Stress Expression of Heat Shock Proteins

After 24 hours of incubation at 37° C., cells plated at 2×10⁵ cells/ml were treated with various concentrations (1/500, 1/1000, or 1/2000) of C. schoenanthus ethanol extract for 72 hours. The cells were then treated with H₂O₂ at 150 μM for 24 h. H₂O₂ was freshly prepared from stock solution prior to each experiment.

For RNA analysis, total RNA was extracted by use of ISOGEN PB Kit. First-strand cDNAs were synthesized from total RNA by using ReverTraAce® reverse transcriptase and random primer at 65° C. for 5 min and 42° C. for 60 min followed by denaturation of the RNA at 70° C. for 10 min. The resultant cDNAs were then used in quantitative PCR analysis.

Transcription levels of genes of interest were determined by performing quantitative PCR using the Applied Biosystems™ 7500/7500 Fast Real-Time PCR System with the following gene-specific primers for HSP27 or HSP90.

### Animals

Adult ICR-strain male mice (5 weeks of age) weighting 26-30 g served as subjects.

Animals were housed in metal cages in acclimatized room (22° C.) with a dark/light cycle of 12 hours. These animals were given free access to water and a commercial diet.

### In Vivo Experimental Design

Solutions were administered to animals orally via gavage.

After a week-long adaptation period, mice were randomized into control and experimental groups and divided into four groups of 8 animals each. Animals in group 1 were administered distilled water. Animals in group 2 were administered imipramine (positive control) at a dose of 15 mg/kg. Animals in groups 3 and 4 were administered C. schoenanthus ethanol extract at doses of 100 and 200 mg/kg body weight each once daily, respectively.

All animals were deprived of food but not water one hour prior to drug administration. Thus, bioavailability of treatment solution was not affected by differences in stomach contents. At other times, animals had free access to food. The procedure is described in Yi et al. 2009.

### Tail Suspension Test

The tail suspension test (TST) was based on the method of Steru (Steru et al., 1985) with minor modifications. Briefly, a mouse was individually suspended by its tail with a clamp (1 cm distant from the tip) for six minutes in a box (35×70×50 cm) with its head 5 cm from the bottom of the box. Testing was carried out in a darkened room with minimal background noise.

The mouse's duration of immobility was observed during the final three minute interval of the test. C. schoenanthus ethanol extract was administered one hour before a TST and imipramine was administered 30 min before a TST.

Data presented in FIG. 7 are the average of 5 observations ±SEM. * P<0.05 vs. Control group (Student's t-test).

### Forced Swimming Test

The forced swimming test (FST) was conducted as previously described by Porsolt et al. (1977). Briefly, a mouse was placed in a 25 cm tall glass cylinder (14 cm in diameter) containing 15 cm of water maintained at 24±2° C., and forced to swim for 6 min (pre-swimming session). Then the mouse was removed and dried with a towel. 24 hours later, the mouse was again forced to swim in a similar environment for six minute (test session). Immobility duration was recorded using a camera during the last three minutes of the six-minute test session.

In acute treatment studies, C. schoenanthus ethanol extract was administered 1 hour before a test session and imipramine was administered 30 min before a test session.

In a sub-chronic treatment study, C. schoenanthus ethanol extract or imipramine, were administered once a day for 2 weeks and the final treatment occurred 1 hour (for the ethanol extracts) or 30 min (for imipramine) before behavioral tests.

Data presented in FIG. 8 are the average of 5 observations ±SEM. * P<0.05 vs. Control group (Student's t-test).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for treating or ameliorating emotional-psychological stress in a subject in need thereof, the method comprising administering to the subject a composition comprising a Cymbopogon schoenanthus ethanol extract as an active ingredient.

2. The method of claim 1, wherein the emotional-psychological stress has one or more symptoms of abnormal blood pressure, agitation, anger, anxiety, confusion, depression, distractibility, exhaustion, fear, insecurity, insomnia, irritability, lack of sexual desire, loss of appetite, low self esteem, menstrual issues, migraine, mood swings, nervousness, panic attacks, rashes, sexual dysfunction, skin eruptions, or social withdrawal.

3. The method of claim 1, wherein the ethanol extract is obtained from a root, a stem, or a blade from a C. schoenanthus plant.

4. The method of claim 1, wherein the composition is formulated as a food product or as a dietary supplement.

5. The method of claim 4, wherein the food product is a beverage, a bread, a candy, a cereal, a chocolate, a coffee, a condiment, a cookie, a cracker, an energy drink, a gel, an ice cream, a jelly, a juice, a milk-containing beverage, a nutritional bar, a pasta, a paste, a processed fruit, a processed grain, a processed meat, a processed vegetable, a pudding, a snack bar, a soft drink, a tea, or a yogurt.

6. The method of claim 4, wherein the dietary supplement is a capsule, a concentrate, a dry syrup, a granule, a liquid gel, a liquid, a lozenge, a paste, a pill, a powder, a softgel, a syrup, or a tablet.

7. The method of claim 1, wherein the C. schoenanthus ethanol extract comprises between 1/10 and 1/10,000 of the composition.

8. The method of claim 7, wherein the C. schoenanthus ethanol extract comprises between 1/100 and 1/2,000 of the composition.

9. The method of claim 8, wherein the C. schoenanthus ethanol extract comprises between 1/500 and 1/1,000 of the composition.

10. The method of claim 9, wherein the C. schoenanthus ethanol extract comprises 1/500 of the composition.

11. The method of claim 9, wherein the C. schoenanthus ethanol extract comprises 1/750 of the composition.

12. The method of claim 9, wherein the C. schoenanthus ethanol extract comprises 1/1,000 of the composition.

13. The method of claim 1, wherein the C. schoenanthus ethanol extract is evaporated to produce a dried C. schoenanthus ethanol extract before being included in the composition.

14. The method of claim 13, wherein the composition comprises between 1 mg and 1,000 mg of the dried C. schoenanthus ethanol extract per kg of the subject.

15. The method of claim 14, wherein the composition comprises between 5 mg and 500 mg of the dried C. schoenanthus ethanol extract per kg of the subject.

16. The method of claim 15, wherein the composition comprises between 10 mg and 300 mg of the dried C. schoenanthus ethanol extract per kg of the subject.

17. The method of claim 16, wherein the composition comprises between 25 mg and 200 mg of the dried C. schoenanthus ethanol extract per kg of the subject.

18. The method of claim 17, wherein the composition comprises between 50 mg and 100 mg of the dried C. schoenanthus ethanol extract per kg of the subject.

19. The method of claim 1, wherein the subject is a human.

20. A method for treating or ameliorating emotional-psychological stress in a human in need thereof, the method comprising administering to the human a tea comprising a dried Cymbopogon schoenanthus ethanol extract as an active ingredient.
